(19) United States
(12) Patent Application Publication
(10) Pub. No.: US 2022/0409630 A1
(43) Pub. Date: Dec. 29, 2022

(54) BIMATOPROST 0.01% SOLUTION COMPOSITIONS FOR THE TREATMENT OF OCULAR HYPERTENSTION

(71) Applicant: RAFARM SA, Athens (GR)

(72) Inventors: Patrizia CHETONI, London, Greater London (GB); Susi BURGALASSI, London, Greater London (GB); Daniela MONTI, London, Greater London (GB); Silvia TAMPUCCI, London, Greater London (GB); Giulia FALCONE, London, Greater London (GB); Christina MATSIGKOU, London, Greater London (GB)

(21) Appl. No.: 17/755,492

(22) PCT Filed: Nov. 2, 2020
(86) PCT No.: PCT/IB2020/060277

§ 371 Date: Apr. 29, 2022

(30) Foreign Application Priority Data
Oct. 31, 2019 (IT) 102019000020190
Dec. 20, 2019 (IT) 102019000024961

(51) Int. Cl. A61K 31/5575; A61K 9/00; A61K 47/18; A61K 47/10

(52) U.S. Cl. CPC A61K 31/5575; A61K 9/0048; A61K 47/186; A61K 47/183; A61K 47/10

(57) ABSTRACT

The invention relates to topical ophthalmic preparations in eye drops containing bimatoprost as an active ingredient at the concentration of 0.1 mg/ml, wherein the presence of benzalkonium chloride is limited to the indispensable minimum commonly required for its activity as an antimicrobial preservative, i.e. from 50 at 80 ppm, and the bioavailability of the product is ensured by the addition of specific non-ionic surfactants, i.e., poloxamers.

### FIELD OF THE INVENTION

The present invention relates to compositions of bimatoprost 0.01% in solution, i.e. containing the active ingredient at a concentration of 0.1 mg/ml, for the topical treatment of ocular hypertension. More particularly, the invention relates to bimatoprost eye drops compositions containing the active ingredient at a concentration of 0.1 mg/ml, wherein the presence of benzalkonium chloride is limited to the minimum necessary concentration commonly envisaged for performing its activity as an antimicrobial preservative. In the proposed formulations, the bioavailability of bimatoprost is ensured by concentrations of less than 0.1% of specific non-ionic surfactant agents, namely, the poloxamers.

### Background of the Invention

Bimatoprost is a prostaglandin analogue, specifically a synthetic prostamide, structurally related to prostaglandin F_{2α} (PGF_{2α}). The molecule is obtained from the substitution of the carboxylic acid group of PGF_{2α} with an electrochemically neutral substituent, and has the following structural formula:

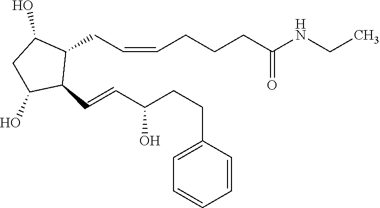 [SMILES: CCNC(=O)CCC/C=C\C[C@@H]1[C@@H](/C=C/[C@@H](O)CCc2ccccc2)[C@H](O)C[C@@H]1O]

Since the carboxylic acid group is fundamental for the interaction of the molecule with the receptor sensitive to PGF_{2α} (FP receptor), bimatoprost does not show any significant pharmacological activity on this receptor. Its mode of action appears to be similar to that of prostamide F_{2α} (PGF_{2α}1-ethanolamide), a naturally occurring substance deriving from anandamide through a pathway involving cyclooxygenase 2 (COX-2), but not COX-1. This pathway leads to the formation of endogenous lipid amides that lower intraocular pressure (IOP), and can be seen as acting in parallel with the arachidonic acid cascade which leads to prostaglandin synthesis.

Prostaglandin analogues are the most recent drug group for the topical treatment of open-angle glaucoma. Instead of reducing the production of aqueous humour by ciliary bodies (as β-blockers or carbonic anhydrase inhibitors), these products lower the IOP by increasing the outflow of aqueous humour through the uveoscleral pathway. In particular, it was found that bimatoprost reduces IOP by increasing uveoscleral and trabecular outflow, and for this reason it is particularly suited to the administration to the eye in the form of eye drops.

Bimatoprost is used as an active ingredient in ophthalmic products for the treatment of glaucoma and ocular hypertension. The first medicinal product based on bimatoprost, marketed under the name Lumigan® (Allergan, Inc.) had a concentration of active ingredient of 0.03% (0.3 mg/ml) and contained benzalkonium chloride (BAK) as an anti-microbial preservative, with a concentration of 0.005% (50 ppm).

Subsequently, a modified version of Lumigan® was put on the market corresponding to the invention described in the patent EP 1753434 (Improved bimatoprost ophthalmic solution). In said patent, the concentration of bimatoprost is reduced to 0.01% and the concentration of BAK is increased to 200 ppm (0.02%). Specifically, Example 4 and FIG. 2 of the patent show that the in vitro apparent trans-corneal permeability (Pₐₚₚ) of corneal cell cultures is greatly enhanced by using lower concentrations of bimatoprost accompanied by significant levels of benzalkonium chloride, possibly in combination with EDTA. It is evident that the aim of such a modification was to increase the bioavailability of the active ingredient, by causing, after the instillation of the eye drops, higher levels of bimatoprost to be able to cross the cornea thus reaching the aqueous humour. In this way, it has been possible to reduce the concentration of active ingredient in the drug from 0.3 mg/ml to 0.1 mg/ml, while achieving a comparable pharmacological efficacy.

From the foregoing, it is clear that the technical problem involved in the development of a new pharmaceutical product based on bimatoprost eye drops, as is the case with many topical ophthalmic products, is to achieve an adequate bioavailability of the active ingredient in internal ocular tissues.

After the topical application of an eye drop, the medicinal solution mixes with the tear fluid and then it is diluted. Furthermore, the contact time of the drug with ocular tissues is relatively short (1-2 min.), mainly due to the leakage of the solution instilled from the pre-corneal area into the lacrimal duct. The ocular absorption of the drug from the lacrimal fluid to the anterior ocular tissues is determined by two main factors: the ability of the drug to permeate through the cornea, and the contact time of the product with the ocular tissues. On the basis of these two principles, various approaches have been proposed to improve the availability of an ophthalmic drug in the anterior chamber of the eye. For example, the use of enhancers has been proposed to increase the permeation of the drug through the cornea, or the use of mucoadhesive/thickening agents has been proposed to increase the residence time of the applied dose in the pre-corneal area.

Topical ophthalmic drugs are generally absorbed through the corneal epithelium via the paracellular pathway (i.e. through the interstices between the epithelial cells left free by the intercellular junctions) or the transcellular pathway (i.e. through the body of the cells themselves). The hydrophilic character of a drug plays a key role in establishing the predominant route of absorption: while hydrophilic drugs are mainly absorbed through the paracellular pathway, the less hydrophilic or hydrophobic drugs are absorbed mainly via the transcellular pathway. The mechanism by which enhancers improve the transport of drugs through the cornea can involve both the paracellular and transcellular pathways.

From the public documentation accompanying both the first and subsequent marketing authorisations of Lumigan® (respectively No. C(2002)1033 of Mar. 8, 2002 and No. C(2006)1816 of Apr. 27, 2006), the potential toxicity of benzalkonium chloride for corneal tissues was already known. For this reason, the following remark is reported in the Summary of Product Characteristics (SmPC, Section 4.4) annexed to the first marketing authorisation: “It has been reported that the Benzalkonium chloride, commonly used as a preservative in ophthalmic products, causes punctate keratopathy and/or toxic ulcerative keratopathy. As Lumigan contains benzalkonium chloride, clinical monitoring is required in patients suffering from dry eye or with impaired cornea who make frequent or prolonged use of the drug”. This observation is much more stringent in the SmPC annexed to the second marketing authorisation, stating as follows: “Since LUMIGAN 0.1 mg/ml contains 200 ppm of benzalkonium chloride (four times the concentration present in bimatoprost 0.3 mg/ml eye drops), it should be used with caution in patients with dry eye or whose cornea may be compromised, and in patients who use different eye drops containing benzalkonium chloride. Moreover, for these patients clinical monitoring is necessary in the event of prolonged use of the drug”.

It is also known from the EMEA documentation published on bimatoprost (Assessment report for Lumigan, European Medicines Agency, London, 7 Jan. 2010, page 31) that bimatoprost exploits the paracellular pathway of penetration, made more efficient by BAK. The latter enhances the ocular permeation of drugs by transiently relaxing the tight epithelial junctions, which temporarily open the paracellular pathway to drug absorption. Therefore, the penetration of bimatoprost significantly benefits from an increase in BAK, and this approach has become the formulation strategy for Lumigan 0.01%. However, as noted, while the prolonged application of excessive levels of BAK can rapidly compromise the entire cornea, the mechanism of action of this agent as a permeation enhancer remains unclear. In particular, it is not clear whether BAK facilitates the transcellular or the paracellular pathway for bimatoprost, or promotes both mechanisms.

In the light of the foregoing, the present invention aims at developing new topical ophthalmic formulations based on bimatoprost as the active ingredient, which formulations are indicated for the treatment of ocular hypertension, in particular of chronic open-angle glaucoma, and allow to improve the bioavailability of the active ingredient, so as to make it possible to use it at the concentration of 0.1 mg/ml without the risks and disadvantages associated with the use of high levels of benzalkonium chloride as a corneal permeation enhancer.

### SUMMARY OF THE INVENTION

In the studies of the present invention, various possible approaches were taken into consideration to improve, on the one hand, the ability of bimatoprost to permeate through the cornea and, on the other, to prolong the contact time of the drug with the corneal surface. It has been considered that some pharmaceutical excipients with surface-active properties are capable of opening the tight junctions of the corneal epithelium. Thus, at low concentrations, such surface-active agents are incorporated in the lipid double-layer, disarranging the membrane and leading to penetration through the cornea into the anterior chamber of the eye.

Another approach to improve transcellular transport by interacting with the components of cellular membranes consists in the use of inclusion agents such as cyclodextrins, for example, hydroxypropyl beta-cyclodextrin (β-W7 in the following), and of non-ionic surfactants such as polysorbate 20 (TW20), ethoxylated hydrogenated castor oil (EL, Kolliphor EL; RH 40, Kolliphor RH40) and d-α-tocopheryl polyethylene glycol 1000 succinate (TPGS, Vitamin E TPGS).

Viscosity-increasing polymers are usually added in ophthalmic solutions to obtain a higher viscosity, which results in a slower elimination from the pre-ocular area and leads to an improved pre-corneal residence time, and therefore to an increased penetration of the drug through the cornea into the anterior chamber of the eye. In general, viscosifying vehicles can increase contact times, but are often unsuitable for producing a prolonged effect.

While increasing the viscosity of an ophthalmic formulation for drug delivery purposes helps to slow down the rapid dilution and drainage caused by the tear film turnover, it is also true that highly viscous solutions can cause transient blurring of vision during instillation and are subject to inaccurate dosing.

An alternative approach involves gelling systems that undergo a phase transition from liquid to gel following exposure to certain physiological conditions, such as ionic strength, temperature or pH. Polymers used for this purpose include poloxamers (such as poloxamer 407 (P407), and poloxamer 188 (P188)), also known under the trade name Pluronic®. The latter are non-ionic three-block copolymers, with a central hydrophobic block of polyoxypropylene and two terminal hydrophilic blocks of polyoxyethylene. Poloxamers organize to form micelles and establish intramolecular hydrogen bonds, thus promoting gelation as a function of temperature.

The use of poloxamers in the release of ophthalmic drugs was reported in 1998 (Edsman K. and Carlfors J., Rheological evaluation of poloxamer as an in situ gel for ophthalmic use, Eur. J. Pharm. Sci., 1998, 6: 105). An increasing concentration of poloxamer has resulted in a high and increasing elasticity of the gels and a decreasing sol-gel transition temperature. Contact time increased with higher concentrations of poloxamer, which could be explained and correlated with the rheology of the poloxamer solution/gel mixed with simulated tear fluid. More recent literature data have reported combinations of poloxamers with mucoadhesive polymers for ophthalmic release.

For example, PCT application No. WO2014/204791 (GNT LLC) discloses ophthalmic lipophilic and hydrophilic drug delivery formulations, one of which contains 0.03% bimatoprost, poloxamer in an amount of 5.5% and BAK of 200 ppm as well.

Generally, however, the concentration of P407 was above 10% (Giuliano E. et al., Mucosal Applications of Poloxamer 407-Based Hydrogels: An Overview, Pharmaceutics 2018, 10 (3), 159).

Furthermore, the ophthalmic use of a combination of poloxamer 407 and Tween 80 (polysorbate 80) in the presence of polyacrylic acid for the administration of fluconazole has been reported in the literature. The resulting in situ gel appears to be a better alternative compared to eye drops (Lihong w. et al., Thermoresponsive ophthalmic poloxamer/tween/carbopol in situ gels of a poorly water-soluble drug fluconazole: preparation and in vitro-in vivo evaluation, Drug Dev. Ind. Pharm., 2014, 40:10, 1402-1410). In these systems the concentration of P407 was 15-20% and the concentration of TW80 (polysorbate 80) was between 0.5% and 1.0% in the various formulations.

In addition to the tight junctions expressed by the corneal epithelium and the barriers limiting the passage through the cornea, recently also the efflux pumps such as glycoprotein P (P-gp) and the proteins associated with multi-drug resistance (MRP) have been shown to have a role in limiting the ocular bioavailability. A variety of efflux transporters are expressed on human corneal epithelium, including multi-drug resistance pumps such as P-gp, BCRP and various MRP isoforms. In vitro and ex vivo experiments have shown that bimatoprost is a substrate for MRP1, MRP2, MRP5 and P-gp, and indicate that permeation through the cornea is limited due to active efflux. Since the human corneal epithelium has been functionally characterized for the presence of MRP and P-gp, the efflux of bimatoprost from the corneal epithelium could play a role in limiting the ocular bioavailability of the same. Furthermore, prostaglandin analogues, including bimatoprost, are administered at a relatively low concentration, thus making efflux a factor of high clinical relevance.

Starting from the above considerations, according to the present invention, combinations of excipients have been selected and further studied, in order to obtain a trans-corneal bioavailability of 0.01% bimatoprost of the same level as the bioavailability obtainable with the use of concentrations of 200 ppm benzalkonium chloride, while maintaining the concentration of this agent at lower levels (50-80 ppm) which are useful for an antimicrobial action thereof.

According to the present invention it has been surprisingly found, contrary to what is suggested by the prior art, that it is possible to formulate bimatoprost in 0.01% eye drops with no more than 80 ppm of benzalkonium chloride, while obtaining a bioavailability comparable with that of Lumigan® 0.1% if a poloxamer, or a mixture of several different poloxamers, is added to the composition. The overall poloxamer concentration must be between 0.005% w/v and 0.1% w/v to achieve equivalent bioavailability to Lumigan 0.1%. Optionally, poloxamer may be in combination with 0.05-0.15% w/v EDTA and/or in combination with 0.01-0.15% w/v polysorbate or Tween (respectively, common and commercial name of ethoxylated sorbitan esters), such as polysorbate 20 or polysorbate 80. The addition of these additional agents will further improve the corneal permeability of bimatoprost.

Other objects and advantages of the present invention will become apparent to those skilled in the art in view of the following detailed description.

### DETAILED DESCRIPTION OF THE INVENTION

The present invention specifically provides an ophthalmic composition in eye drops for use in the treatment of ocular hypertension and glaucoma, which composition comprises from 0.008% to 0.012% w/v of bimatoprost as the active ingredient, from 0.005% to 0.1% w/v overall of one or more poloxamers (or Pluronic®), and an antimicrobial agent consisting of benzalkonium chloride, at a concentration of from 50 to 80 ppm, in an aqueous vehicle.

As an alternative to benzalkonium chloride, it is possible to use other preservatives suitable for ophthalmic administration, in appropriate concentrations. Examples are quaternary ammonium salts, such as benzethonium chloride, benzoxonium chloride, or polydronium chloride (Polyquad, Polyquaternium-1), alcohols, such as chlorobutanol, benzyl alcohol (phenylmethyl alcohol), guanidine derivatives, such as polyhexamethylene biguanide, chlorhexidine digluconate, alkyl-mercury salts of thiosalicylic acid, such as thiomersal, phenylmercuric nitrate, phenylmercuric acetate, phenylmercuric borate, parabens, such as methylparaben (methyl para-hydroxybenzoate) or propylparben (propyl para-hydroxybenzoate), stabilized oxychloro complexes such as Purite® or sorbic acid. The concentrations in which each of these preservatives can be present are those provided by the various regulatory bodies for ophthalmic preparations (see, for example, “Pharmaceutical Preformulation and Formulation”, second Ed., 2009, edited by Mark Gibson, Informa Healtcare). Where appropriate, a sufficient amount of preservative is added to the ophthalmic composition to ensure protection against secondary contaminations.

The ophthalmic solution according to the present invention may also contain stabilizing agents to inhibit decomposition of the active substance in the ophthalmic solution. Preferred examples of stabilizing agents are ethylenediaminetetraacetic acid and salts thereof, such as disodium edetate, and dibutylhydroxytoluene. Also other stabilizing agents, such as ascorbic acid and esters thereof, sodium hydrogensulfite, cysteine hydrochloride, citric acid, tocopherol acetate, soybean lecithin, natural vitamin E, tocopherol, butylhydroxyanisole, 1,3-butylene glycol, propyl gallate, 2-mercaptobenzimidazole and oxyquinoline sulphate, may be used. According to a specific embodiment, the formulation of the present invention comprises EDTA (ethylenediaminetetraacetic acid) in a concentration ranging from 0.05 to 0.15% w/v, as stabilizing agent against oxidative degradation. The preferred concentration of EDTA in the ophthalmic solution of the present invention is 0.1% w/v.

According to another specific embodiment the composition of the present invention contains at least one poloxamer as polymeric excipient of the composition, and preferably said components are present in an overall concentration equal to 0.01% w/v.

Specifically, the poloxamers are preferably selected from poloxamer 188 (P188), poloxamer 407 (P407) and mixtures thereof.

According to a further specific embodiment of the present invention, the eye drops ophthalmic formulation for use in the treatment of ocular hypertension and glaucoma can also comprise from 0.01 to 0.15% w/v of a polysorbate (Tween), or a mixture of two or more of the same, such as polysorbate 20, polysorbate 40, polysorbate 60, etc. The polysorbate preferably used in the formulation of the present invention is polysorbate 20 (Tween 20).

In all of the embodiments listed above, bimatoprost can preferably be present in a concentration of 0.01% w/v, and benzalkonium chloride can be present in a preferred concentration of 50 ppm.

As in any other composition for topical eye drops, the formulation of the invention may also include the common excipients used to maintain an osmolarity and a pH suitable for ophthalmic administration. In particular, a buffer system, preferably based on disodium phosphate heptahydrate and citric acid, as well as an isotonizing agent such as sodium chloride, are present in the composition. The composition is brought to a pH between 7.0 and 7.5 by adjusting it with NaOH/HCl.

Other customary ophthalmically acceptable excipients and additives known to the person skilled in the art may be comprised in an above composition, for example one or more of any of the following; carriers, stabilizers, solubilisers, tonicity enhancing agents, buffer substances, preservatives, viscosity enhancing agent, and other excipients. Such compositions are prepared in a manner known, for example by mixing the active ingredients with the corresponding excipients and/or additives to form corresponding ophthalmic compositions.

Carriers that could be used in accordance to the present invention are those suitable for topical administration, and are, apart from water, mixtures of water and water-miscible solvents, such as C₁ to C₇ alkanols, vegetable oils or mineral oils comprising from 0.5 to 5% by weight hydroxyethylcellulose, ethyl oleate, carboxymethyl-cellulose, polyvinyl-pyrrolidone and other non-toxic water-soluble polymers for ophthalmic uses, such as, for example, cellulose derivatives, such as methylcellulose, alkali metal salts of carboxy-methylcellulose, hydroxymethylcellulose, hydroxyethylcellulose, methylhydroxypropyl-cellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as hyaluronic acid, polyvinyl alcohol, polyvinylpyrrolidone, polyvinyl methyl ether, polyethylene oxide, preferably cross-linked polyacrylic acid, such as neutral Carbopol, or mixtures of those polymers. Preferred carriers are water, cellulose derivatives, such as methylcellulose, alkali metal salts of carboxymethylcellulose, hydroxymethylcellulose, hydroxyethylcellulose, methylhydroxypropylcellulose and hydroxypropylcellulose, neutral Carbopol, or mixtures thereof.

Solubilisers that could be used for an ophthalmic composition of the present invention are, for example, tyloxapol, fatty add glycerol polyethylene glycol esters, fatty add polyethylene glycol esters, polyethylene glycols, glycerol ethers, polyoxyethylene stearates, cyclodextrins, polyoxyethylene hydrogenated castor oils, macrogol 15 hydroxystearate, polyethylene-propylene glycol copolymers, polyoxyethylene sorbitan fatty add esters, or mixtures of those compounds. A specific example of an especially preferred solubiliser is poloxamer, preferably poloxamer 407 or 188. Another preferred solubiliser may be a reaction product of castor oil and ethylene oxide, for example the commercial products Cremophor EL® or Cremophor® RH 40. Reaction products of castor oil and ethylene oxide have proven to be particularly good solubilisers that are tolerated extremely well by the eye. Other preferred solubilisers are tyloxapol and polysorbate, preferably polysorbate 20 or polysorbate 80.

Examples of buffer substances are acetate, ascorbate, borate, hydrogen carbonate/carbonate, citrate, gluconate, lactate, phosphate, propionate and TRIS (tromethamine) buffers. Phosphate and citrate buffer are preferred buffers. The amount of buffer substance added is, for example, that necessary to ensure and maintain a physiologically tolerable pH range.

The pH range is typically in the range of from 5 to 9, preferably from 5.2 to 8.5 and more preferably from 5.5 to 8.2. Ideally, ophthalmic solutions should have the same pH as the lacrimal fluid (7.4), but pH values from 7 to 9 are tolerated by the eye without marked irritation. The buffer capacity of the lacrimal fluid (0.01 ml) should not be exceeded due to increased tear production and eye movement, resulting in increased eye drop clearance.

The lacrimal fluid is isotonic (i.e. has the same tonicity) with blood with 287 mOsm/l. Ideally, an ophthalmic solution should have the same tonicity values as the lacrimal fluid, but the eye can tolerate a rather broad range of tonicity from ˜205-683 mOsm/l (USP, 1995). Preferably, the osmolality is 300-500 mOsm/l. The tonicity is adjusted by the use of suitable agents as disclosed herein. The osmolality in normal tear fluid is from 310 to 340 mOsmol/kg, and ideally the formulation has an osmolality that is inside this range and the amounts of the tonicity ingredients are adjusted accordingly. In certain cases, the osmolality might be desired to be higher (hyper osmolality formulation) or lower (hypo osmolality formulation) and this depends upon other factors such as pH and colloidal osmolality effects caused by any viscosity agent added into the formulation. The osmolality may be measured using a freezing-point depression osmometer, following the procedure set out in the US Pharmacopeia, USP 34, National Formulary 29, 2011, chapter 785. Tonicity enhancing agents are, for example, ionic compounds, such as alkali metal or alkaline earth metal halides, such as, for example, CaCl2, KBr, KCl, LiCl, NaI, NaBr or NaCl, or boric add. Non-ionic tonicity enhancing agents are, for example, urea, glycerol, sorbitol, mannitol, propylene glycol, or dextrose. For example, sufficient tonicity enhancing agent is added to impart to the ready-for-use ophthalmic composition an osmolality of approximately from 50 to 1000 mOsmol/kg, preferred from 100 to 400 mOsmol/kg, more preferred from 200 to 400 mOsmol/kg and even more preferred from 250 to 350 mOsmol/kg, ideally 280-300 mOsmol/kg.

The surface tension of the lacrimal fluid ranges from 40 to 50 mN/m. Low surface tension provides good spreading effect on the cornea, possibly improving the contact between the drug and corneal epithelium. Preferably, the surface tension is from 20 to 60 mN/m. The surface tension is adjusted using wetting agents as is customary. Such wetting agents include excipients that also act as solubilisers or preservatives.

Any pharmaceutically acceptable packaging material may be used, preferably a packaging material suitable for administration of an ophthalmic product. Pharmaceutically acceptable packaging materials include but are not limited to materials made of polyethylene, preferably low density polyethylene or high density polyethylene, polypropylene, polystyrene, polycarbonate, polyesters, preferably polyethylene terephthalate or polyethylene naphthalate, nylon, polyvinyl chloride, cyclo-olefin copolymers and other materials known to those skilled in the art. Preferred containers include a bottle with a dropper and a cap or single-use vials or ampoules.

The present disclosure also relates to a method of treating ocular hypertension and/or glaucoma, which method comprises topically applying to the exterior surface of the eye a composition comprising from 0.008% to 0.012% w/v of bimatoprost as the active ingredient, from 0.005% to 0.1% w/v overall of one or more poloxamers (or Pluronic®), and an antimicrobial agent consisting of benzalkonium chloride, at a concentration of from 50 to 80 ppm, in an aqueous vehicle.

The present disclosure also relates a composition comprising from 0.008% to 0.012% w/v of bimatoprost as the active ingredient, from 0.005% to 0.1% w/v overall of one or more poloxamers (or Pluronic®), and an antimicrobial agent consisting of benzalkonium chloride, at a concentration of from 50 to 80 ppm, in an aqueous vehicle for use in treating ocular hypertension and/or glaucoma.

As used herein, the term “topical” means applied to the external surface of the eye, i.e. onto the cornea.

In view of the clinical indication for glaucoma and/or ocular hypertension, the treatment described herein is typically used for an extensive period often for life, wherein the bimatoprost containing composition is administered over an extended period of time. Therefore, the lower amounts of benzalkonium chloride found in the present composition are particularly important. It is preferred that the composition is applied daily, preferably once a day in the evening or in the morning. Optionally, it may be applied twice a day—once in the morning and once in the evening. The treatment should be applied for the same duration applicable to the other known therapies of open angle glaucoma or ocular hypertension based on bimatoprost.

### EXAMPLES

The following examples illustrate preferred embodiments in accordance with the present invention without limiting the scope or spirit of the invention.

### Example 1

Formulations referred to as P188 0.01 BAK 50

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P188 | 0.01 | |
| NaOH/HCl 1N | q.s. at pH = 7.0-7.5 | |
| BAK | | 50 |

### Example 2

Formulations referred to as P407 0.01 BAK 50

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P407 | 0.01 | |
| NaOH/HCl 1N | q.s. at pH = 7.0-7.5 | |
| BAK | | 50 |

### Example 3

Formulations referred to as P407 0.005 BAK 50

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P407 | 0.005 | |
| NaOH/HCl 1N | q.s. at pH = 7.0-7.5 | |
| BAK | | 50 |

### Example 4

Formulations referred to as P188 0.01/EDTA 0.1 BAK 50

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P188 | 0.01 | |
| NaOH/HCl 1N | q.s. at pH = 7.0-7.5 | |
| BAK | | 50 |
| EDTA | 0.1 | |

### Example 5

Formulations referred to as P407 0.01/EDTA 0.1 BAK 50

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P407 | 0.01 | |
| NaOH/HCl 1N | q.s. at pH = 7.0-7.5 | |
| BAK | | 50 |
| EDTA | 0.1 | |

### Example 6

Formulations referred to as P188 0.01/TW20 0.1 BAK 50

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P188 | 0.01 | |
| NaOH/HCl 1N | q.s. at pH = 7.0-7.5 | |
| TW 20 | 0.1 | |
| BAK | | 50 |

### Example 7

Formulations referred to as P407 0.01/TW20 0.1 BAK 50

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P407 | 0.01 | |
| NaOH/HCl 1N | q.s. at pH = 7.0-7.5 | |
| TW 20 | 0.1 | |
| BAK | | 50 |

### Example 8

Formulations referred to as P188 0.01 BAK 80

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P188 | 0.01 | |
| NaOH/HCl 1N | q.s. at pH = 7.0-7.5 | |
| BAK | | 80 |

### Example 9

Formulations referred to as P407 0.01 BAK 80

| | Concentration in water | |
|---|---|---|
| Ingredients | % w/v | ppm |
| Bimatoprost | 0.01 | |
| Citric acid monohydrate (or anhydrous) | 0.0050-0.020 | |
| Dibasic sodium phosphate heptahydrate | 0.100-0.400 | |
| (Na₂HPO₄•7H₂O) | | |
| Sodium chloride | 0.5-0.85 | |
| Poloxamer P407 | 0.01 | |
| NaOH/HCl 1N | q.s. at pH = 7.0-7.5 | |
| BAK | | 80 |

In the following technological study some of the experimental results that led to the present invention are reported.

### BRIEF DESCRIPTION OF THE FIGURES

The specific features of the invention will be more evident with reference to the accompanying drawings, in which:

FIGS. 1 and 2 respectively show in histogram form the apparent permeability through the cornea (unit: 10⁻⁶ cm/s) and the resistance value (unit: Ohm) of the various bimatoprost formulations tested in the experimental study reported below, which formulations include the compositions according to the present invention;

FIGS. 3 and 4 show in histogram form the apparent permeability through the cornea (unit: 10⁻⁶ cm/s) of different bimatoprost formulations, including the compositions of the present invention, for the purpose of comparing the effects of, respectively, the addition of different surfactants or complexing agents and the addition of EDTA.

### EXPERIMENTAL STUDY

The aim of the study was to obtain experimental data to create new ophthalmic formulations for topical application containing 0.01% bimatoprost as an active ingredient that were capable of presenting a therapeutic activity similar to that of Lumigan® 0.01% but with a low concentration of benzalkonium chloride, of the order of 50-80 ppm, with the simple function of preservative.

As known, the reference product Lumigan® 0.01% eye drops (Allergan) contains 200 ppm of benzalkonium chloride, which in this concentration exercises two different actions: it preserves the solution from microbial contamination and acts as a promoter for the permeation of bimatoprost through the cornea.

To support the study of a new formulation of bimatoprost in 0.01% solution with about 50-80 ppm of benzalkonium chloride, permeation studies were conducted through EpiCorneal corneal tissues (COR-100, Mattek) and transcorneal permeation studies ex vivo through isolated rabbit corneas, to verify the ocular absorption of bimatoprost.

Main excipients chosen for all formulations
a) Buffer solution: a buffer solution based on disodium hydrogen phosphate (Emprove®, Merck) with citric acid (Citric Acid, SA Citrique Beige N.V.) was chosen, with a pH between 7.2 and 7.4. The phosphate concentration corresponded to that used in the reference product.
   Buffer composition: Na₂HPO₄ heptahydrate (10 mM) and citric acid monohydrate (PBC).
b) Isotonizing agent: Sodium chloride (Emprove®, Merck) was chosen as an isotonizing agent for all formulations.
   The concentration of NaCl was in the range between 0.83% w/v and 0.65% w/v.
c) Antibacterial agent: benzalkonium chloride (BAK, Novo Nordisk Pharmatech A/S) was used at different concentrations depending on the type of experimental procedure. In particular, the formulation with 0.01% of BAK (100 ppm) was used for the ex vivo corneal permeation studies, and concentrations of 0.008% w/v (80 ppm) or lower (up to 0.005%, 50 ppm) were used in all formulations for trans-corneal permeation studies in vitro through the COR-100 epicorneal membranes (Mattek).
d) Stabilizing agent: the inclusion of a stabilizing agent could be useful to reduce the oxidative degradation that can be accelerated by the presence of metallic impurities: for this purpose, sodium EDTA was chosen (EDTA, Emprove® Merck).

### Bimatoprost Permeation Study (BMTP) Through EpiCorneal Tissues (COR-100 Mattek)

### Experimental Procedure

Study formulations: all the formulations in the study were prepared in phosphate/citrate buffer and contained, in addition to the excipients, 0.005% w/v (50 ppm) or 0.008% w/v (80 ppm) of benzalkonium chloride (BAK) and 0.01% w/v of bimatoprost. NaCl was chosen as an isotonizing agent and the pH and osmolality values have been measured immediately after the preparation of the formulations.

Commercial preparations Lumigan 0.01% and Lumigan 0.03% used as reference products and two BMTP reference solutions based exclusively on bimatoprost (0.01%) in phosphate/citrate buffer, added with 0.005% of BAK (50 ppm) or 0.008% of BAK (80 ppm) were chosen as control solutions. The reference formulations contained 0.815% w/v of sodium chloride as an isotonizing agent. The buffer solution based on disodium hydrogen phosphate supplemented with citric acid with a pH between 7.2 and 7.4 was chosen. Buffer composition: 0.268% w/v of Na₂HPO₄ heptahydrate and 0.0128% w/v anhydrous citric acid. The formulations contained 0.815% w/v of sodium chloride as an isotonizing agent.

Evaluation of apparent permeability: An exactly weighted amount of BMTP corresponding to 0.01% w/v was dissolved in at least 85% of the theoretical amount of ultrapure water (MilliQ water, Millipore) required for each preparation, and then appropriate amounts of the other key excipients have been added: hydrogen phosphate disodium, citric acid, benzalkonium chloride and sodium chloride. Finally, water was added to the mixture up to volume and 0.815% w/v of NaCl. The formulations were kept under constant agitation for 4-6 hours at room temperature.

The EpiCorneal COR-100 tissues were equilibrated overnight in the culture medium (COR-100 ASY medium) under standard cell culture conditions (SCC, 37° C., 5% CO₂) following the detailed Mattek protocol as reported in the application note. Then, the tissues were transferred to two different 12-well plates and balanced with 500 μL of Krebs Ringer bicarbonate buffer (KRB, pH 7.4) for 30 minutes before the starting of each treatment.

After equilibrating the tissue for 30 minutes in 500 μL of KBR medium, 100 μL of each selected preparation, previously thermostated maintaining the solutions at 37° C. in a water bath, were applied on the surface of the tissue. Then, the well plates were incubated in SCC. A slight agitation of the well plates was used to reduce the possibility that the drug molecule permeated near the basolateral layer of EpiCorneal tissue could hinder drug permeation across the barrier. At predetermined time intervals (0.5, 1, 2, 3, 4 hours), 300 μL of KRB receiving soil were collected and replaced with the same volume of fresh medium.

At the end of the experiment, the donor phase of each well plate was completely withdrawn to analyse the amount of BMTP. Each preparation was evaluated in duplicate.

The steady state flow (J) of BMTP was calculated from the slope of the graphs of BMTP permeate quantity per unit area over time (μg·cm⁻²h⁻¹) and the apparent permeability (Pₐₚₚ, cm·s⁻¹) was obtained according to the following mathematical relation:

Pₐₚₚ=J/(3600×C_{donor})

In any case, the amount of BMTP used for the calculations of flow and apparent permeability of BMTP is the cumulative amount of permeate drug that takes into consideration the drug removed from the receiving compartment.

Evaluation of tissue resistance: resistance measurements on EpiCorneal tissues were performed to assess the integrity of the corneal barrier before the start of permeation studies and at the end of the experiments. The measurements were performed using the EVOM® epithelial voltmeter equipped with a fixed pair of silver/silver chloride rod electrodes. The resistance values have been read directly on the digital display of the instrumentation. The initial resistance (t=0 h; TEER-1) of two different samples in different EpiCorneal well plates was measured after pre-treatment, immediately after filling the wells with 300 μL (donor phase) and 500 μL (receiver phase) of KRB buffer.

Furthermore, tissue resistance was measured for each sample at the end of the permeation study (t=4 h; TEER-2). To perform these measurements at the end of the permeation studies, each well plate was transferred to a new well plate containing 500 μL and 300 μL of fresh KBR in the receiving compartment and in the donor compartment, respectively. The results of the experimentation are summarized in Table 1 below.

TABLE 1
Results of the permeation study through tissue EpiCorneal

| | | | | Apparent | |
|---|---|---|---|---|---|
| | | | | permeability | TEER-2 |
| Name of the | | | Concentration | (average ± d.s.) | (average ± d.s.) |
| formulation | Excipients | BAK | (% w/v) | (10⁶ cm/s) | (Ohm) |
| P188 0.1 BAK 50 | Polox. 188 | 50 | 0.1 | 1.20 ± 0.68 | 632.0 ± 13.0 |
| P188 0.01 BAK 50 | Polox. 188 | 50 | 0.01 | 4.46 ± 0.91 | 650.2 ± 126.3 |
| P188 0.005 BAK 50 | Polox. 188 | 50 | 0.005 | 2.51 ± 0.01 | 657.5 ± 31.2 |
| P188 0.01 BAK 80 | Polox. 188 | 80 | 0.01 | 4.36 ± 0.261 | 528.0 ± 117.4 |
| P407 0.1 BAK 50 | Polox. 407 | 50 | 0.1 | 2.0 ± 0.90 | 660.0 ± 20.0 |
| P407 0.01 BAK 50 | Polox. 407 | 50 | 0.01 | 4.33 ± 0.951 | 534.5 ± 43.2 |
| P407 0.005 BAK 50 | Polox. 407 | 50 | 0.005 | 4.48 ± 1.217 | 601.8 ± 66.3 |
| P407 0.01 BAK 80 | Polox. 407 | 80 | 0.01 | 4.54 ± 0.078 | 541.5 ± 13.4 |
| TW20 0.02 BAK 50 | TW 20 | 50 | 0.02 | 1.23 ± 0.05 | 716.0 ± 22.3 |
| TW20 0.1 BAK 50 | TW 20 | 50 | 0.1 | 2.52 ± 0.20 | 813.3 ± 48.5 |
| TPGS 0.5 BAK 50 | TPGS | 50 | 0.5 | 2.13 ± 0.23 | 604.5 ± 25.4 |
| β-W7 1.0 BAK 50 | β-W7 | 50 | 1.0 | 1.39 ± 0.44 | 696.0 ± 32.9 |
| P188 0.1/TW20 | Polox. 188 | 50 | 0.1 | 0.87 ± 0.05 | 832.0 ± 9.9 |
| 0.02 BAK 50 | TW 20 | | 0.02 | | |
| P188 0.01/TW20 | Polox. 188 | 50 | 0.01 | 1.98 ± 0.17 | 798.0 ± 8.3 |
| 0.02 BAK 50 | TW 20 | | 0.02 | | |
| P188 0.01/TW20 | Polox. 188 | 50 | 0.01 | 3.93 ± 1.85 | 766.5 ± 30.4 |
| 0.1 BAK 50 | TW 20 | | 0.1 | | |
| P188 0.01/EL | Polox. 188 | 50 | 0.01 | 1.81 ± 0.33 | 798.0 ± 8.3 |
| 0.01 BAK 50 | EL | | 0.01 | | |
| P188 0.01/EDTA | Polox. 188 | 50 | 0.01 | 4.56 ± 1.16 | 805.0 ± 35.4 |
| 0.1 BAK 50 | EDTA | | 0.1 | | |
| P407 0.01/TW20 | Polox. 407 | 50 | 0.1 | 4.25 ± 0.23 | 558.5 ± 68.6 |
| 0.1 BAK 50 | TW 20 | | | | |
| P407 0.01/EDTA | Polox. 407 | 50 | 0.01 | 4.56 ± 1.16 | 611.5 ± 0.7 |
| 0.1 BAK 50 | EDTA | | 0.1 | | |
| P188 0.01/P407 | Polox. 188 | 50 | 0.01 | 3.04 ± 0.106 | 596.5 ± 19.1 |
| 0.01 BAK 50 | Polox. 407 | | 0.01 | | |
| P188 0.01/P407 | Polox. 188 | 50 | 0.01 | 3.68 ± 0.49 | 552.5 ± 46.0 |
| 0.005 BAK 50 | Polox. 407 | | 0.005 | | |
| P188 0.01/P407 | Polox. 188 | 80 | 0.01 | 2.86 ± 0.544 | 572.5 ± 12.0 |
| 0.01 BAK 80 | Polox. 407 | | 0.01 | | |
| P188 0.01/P407 | Polox. 188 | 80 | 0.01 | 3.57 ± 0.382 | 555.0 ± 7.1 |
| 0.005 BAK 80 | Polox. 407 | | 0.005 | | |
| P188 0.01/EDTA | Polox. 188 | 80 | 0.01 | 3.84 ± 0.233 | 558.5 ± 68.6 |
| 0.1 BAK 80 | EDTA | | 0.1 | | |
| BMTP 0.01 BAK 80 | | 50 | | 2.81 ± 0.962 | 707.7 ± 105.8 |
| BMTP 0.01 BAK 50 | | 80 | | 3.52 ± 0.601 | 634.5 ± 36.1 |
| Lumigan 0.01% | | 200 | | 4.696 ± 0.764 | 439.1 ± 117.3 |
| Lumigan 0.03% | | 100 | | 3.37 ± 0.59 | 665.5 ± 20.5 |

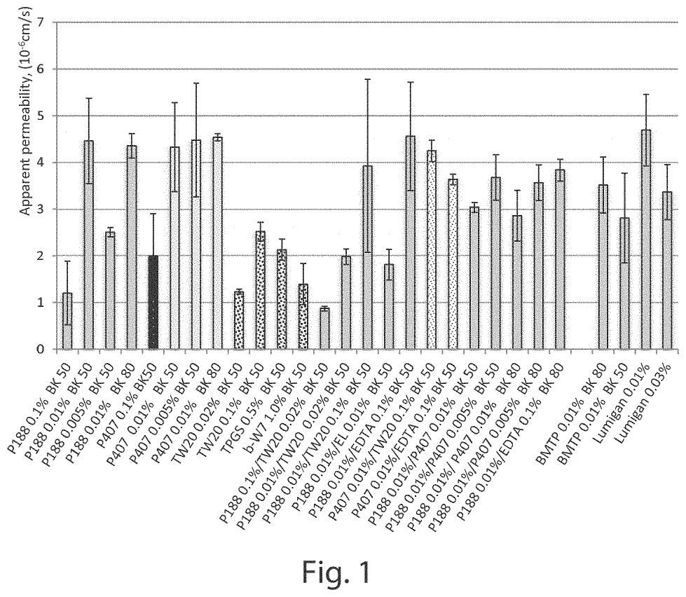

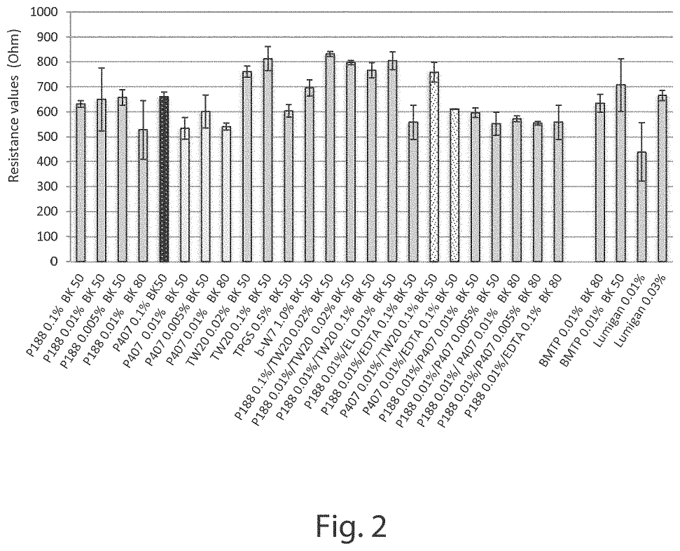

From the data of Table 1, which are generally represented in the form of histograms in FIGS. 1 (apparent permeability) and 2 (resistance values) it can be observed that, surprisingly, the P188 used at 0.01% and containing 50 ppm of BAK showed a higher permeability coefficient than that of the reference solution. Furthermore, the value of Pₐₚₚ was of the same order of magnitude as that calculated for commercial Lumigan at 0.01%, which contains 200 ppm of BAK. The highest Pₐₚₚ coefficient was measured for P188 0.01%: in fact, both concentrations higher (0.1%) and lower (0.005%) of P188 determined a reduction in the apparent permeability of bimatoprost.

The Pₐₚₚ value for the formulations containing P188 0.01% and 80 ppm of BAK was in the same order of magnitude as that calculated for Lumigan 0.01%, which contains 200 ppm of BAK.

All the formulations tested showed higher resistance values than those measured in the presence of 0.01% Lumigan.

It should be noted that a high value of corneal resistance is a fundamental requirement for the ophthalmic formulations in the present study.

A high resistance value was measured for the EpiCorneal tissue before the permeation study (1340 Ohm, for white Epicorneal), while after 4 hours of bimatoprost permeation test, all the treated tissues showed resistance values lower, between 500 and 800 Ohm, except those treated with the commercial 0.01% Lumigan. This reduction in resistance does not appear to be associated with the selected excipients but appears to be caused by the presence of benzalkonium chloride (50 and 80 ppm). In fact, the EpiCorneal fabric treated with the commercial Lumigan 0.01% preparation which contained a higher concentration of benzalkonium chloride, showed the lowest resistance value, up to 35% (439.1 Ohm) of the initial value.

It should be noted that the addition of different concentrations of other non-ionic surfactants (TPGS, TW20) or complexing agents such as hydroxypropyl-8-cyclodextrin (β-W7) in the presence of BAK at 50 ppm did not lead to an increase in Pₐₚₚ of BMTP with respect to the BMTP reference formulation.

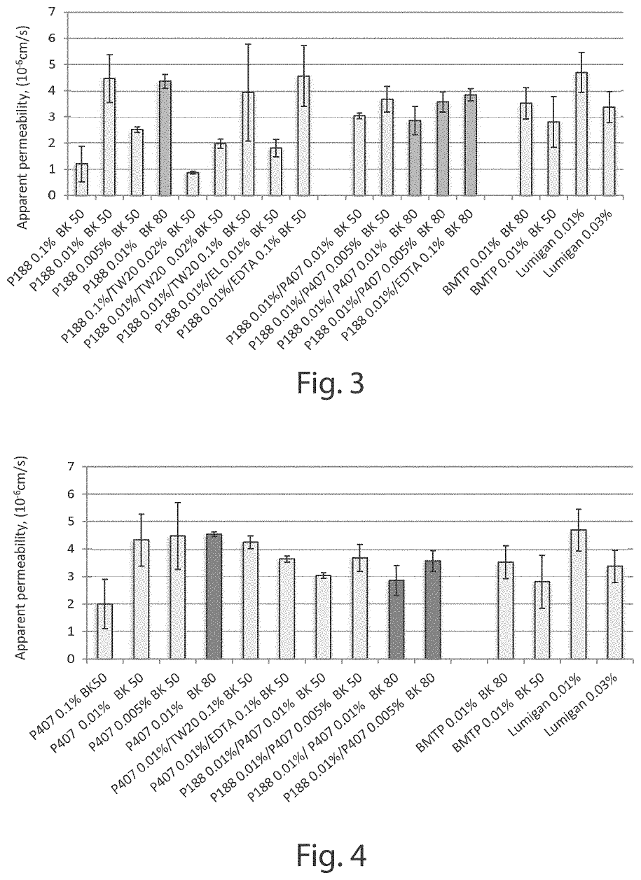

In all cases the Pₐₚₚ values were lower than those calculated for the reference BMTP of 0.01% with 50 ppm of BAK, and for both Lumigan 0.01% containing 200 ppm of BAK and Lumigan 0.03% containing 100 ppm of BAK, as shown more clearly in the histogram of FIG. 3.

As shown more clearly in the histogram of FIG. 4, adding EDTA to the formulation containing P188 0.01% and BAK 50 determines a Pₐₚₚ value similar to that calculated for P188 0.01% BAK 50 in the absence of EDTA.

The addition of other types of poloxamers (e.g. P407 at 0.01% concentration) to the formulation based on 0.01% of P188 and BAK 50 ppm, which represents the most promising concentration capable of favouring the apparent permeability of BMTP, resulted in a reduction in BMTP Pₐₚₚ values even in the presence of different BAK concentrations (50 and 80 ppm).

Similarly, the combination of P188 0.01% with TW 20 0.02% and 0.1%, and P188 0.01% with EL 0.01%, caused a decrease in the apparent permeability of BMTP. The values obtained were lower for all the blends tested, with respect to the commercial product and the references containing up to 80 ppm of BAK (FIG. 4).

### Corneal Permeation Study Ex Vivo

Experimental design: BMTP permeation through isolated rabbit corneas was performed using a plexiglass perfusion apparatus where rabbit corneas (New Zealand) were mounted immediately after animal sacrifice, to separate a donor compartment with volume of 1.0 ml and an acceptor compartment with a volume of 5.0 ml. Both compartments were oxygenated with O₂:CO₂ mixture (95:5) and the experiments were conducted in Ringer's glutathione bicarbonate solution (GBR) at pH 7.2 and 32° C. to maintain corneal viability up to at 5 hours. The formulations were added to the donor compartment, replacing 500 μL of donor solution. At different times (approximately every hour) samples of the receiving solution were collected and analysed by HPLC. The quantitative analysis of the BMTP in the reception phase at different times of sampling and in the donor phase at the end of the experiments was calculated using an appropriate calibration curve.

TABLE 2
Pₐₚₚ values of formulations used for
ex vivo corneal permeability study

| | | BAK | BAK | Pₐₚₚ |
|---|---|---|---|---|
| | | (ppm) | (ppm) | (cm/s) |
| 1 | Lumigan 0.01% | 200 | | 9.10 10⁻⁶ |
| 2 | P188 (0.1%)-TW20 (0.02%) | 100 | | 5.06 10⁻⁶ |
| 3 | P188 (0.1%)-TW20 (0.02%) | | 50 | 0.032 10⁻⁶ |
| 4 | P188 (0.1%)-RH40 (0.5%) | 100 | | 3.04 10⁻⁶ |
| 5 | P188 (0.1%)-TW80 (1.0%) | 100 | | 4.03 10⁻⁶ |

The formulations No. 2, 4 and 5 contained P188 (0.1%) plus TW20 (0.02%) or RH40 (0.5%) or TW80 (1.0%) and the BAK concentration was 100 ppm. Formulation No. 3 had the same composition as formulation No. 2 but contained 50 ppm of BAK.

In all cases the formulations tested did not determine the apparent permeability of Lumigan 0.01%, and a reduction in Pₐₚₚ was detected by reducing the concentration of BAK from 100 ppm to 50 ppm respectively for formulations No. 2 and No. 3.

The present invention has been described with reference to some specific embodiments thereof, but it is to be understood that variations or modifications may be made to it by those skilled in the art without departing from the scope of protection as defined in the appended claims.

1. An eye drops ophthalmic composition for use in the treatment of ocular hypertension and glaucoma comprising from 0.008% to 0.012% w/v of bimatoprost as active ingredient, from 0.005% to 0.1% w/v in total of one or more poloxamers, and an antimicrobial agent consisting of benzalkonium chloride, at a concentration of from 50 to 80 ppm, in an aqueous vehicle.

2. The eye drops ophthalmic composition for the use according to claim 1, further comprising from 0.05 to 0.15% w/v of EDTA.

3. The eye drops ophthalmic composition for the use according to claim 2, wherein the concentration of said EDTA is 0.1% w/v.

4. The eye drops ophthalmic composition for the use according to claim 1, wherein said one or more poloxamers are the only polymeric excipients of the composition.

5. The eye drops ophthalmic composition for the use according to claim 4, wherein said one or more poloxamers are present at a total concentration of 0.01% w/v, and are selected from poloxamer 188, poloxamer 407 and mixtures of the same.

6. The eye drops ophthalmic composition for the use according to claim 1, further comprising from 0.01 to 0.15% w/v of a polysorbate, or of a mixture of two or more polysorbates.

7. The eye drops ophthalmic composition for the use according to claim 6, wherein said polysorbate is polysorbate 20.

8. The eye drops ophthalmic composition for the use according to claim 1, wherein said bimatoprost is present at a concentration of 0.01% w/v, and said benzalkonium chloride is present at a concentration of 50 ppm.

9. The eye drops ophthalmic composition for the use according to claim 1, also comprising a buffer system, preferably based on disodium phosphate heptahydrate and citric acid, an isotonizing agent, such as sodium chloride, said composition being brought to a pH between 7.0 and 7.5 by adjustment with NaOH/HCl.
